(19) United States
(12) Patent Application Publication
(10) Pub. No.: US 2022/0241537 A1
(43) Pub. Date: Aug. 4, 2022

(54) Respiration Aid

(71) Applicant: Worcester Polytechnic Institute, Worcester, MA (US)

(72) Inventor: Elizabeth A. Butler, Auburn, MA (US)

(21) Appl. No.: 17/592,837

(22) Filed: Feb. 4, 2022

Related U.S. Application Data
(60) Provisional application No. 63/145,640, filed on Feb. 4, 2021.

(51) Int. Cl. A61M 16/06; A61D 3/00

(52) U.S. Cl. CPC A61M 16/0672; A61D 3/00

(57) ABSTRACT

An oxygen catheter for veterinary patients includes an integrated catheter, cannula or vessel having integrated lumens for both oxygen and anesthetic such as lidocaine. The oxygen lumen extends into the trachea of the veterinary patient, often a canine, for respiratory aid. The anesthetic lumen is integrated into the same catheter as the oxygen, and terminates prior to the oxygen lumen for administering an anesthetic such as lidocaine around the soft palate to ease discomfort of the inserted catheter. The integration of the respiratory and anesthetic vessels allows a lidocaine drip to accompany the catheterized oxygen supply to medicate the tracheal, pharynx and soft palate regions from the inserted catheter along the path of the oxygen lumen to ease patient discomfort and anxiety for effective respiratory treatment while avoiding more invasive and expensive measures.

### RELATED APPLICATIONS

This patent application claims the benefit under 35 U.S.C. § 119(e) of U.S. Provisional Patent App. No. 63/145,640, filed Feb. 4, 2021, entitled “RESPIRATION AID,” incorporated herein by reference in entirety.

### BACKGROUND

Veterinary interventions can be complicated by an inability to communicate the need for intervention to the patient. Most humans can be advised of the need and effect for a possibly uncomfortable intervention, but canine patients cannot be easily compelled to voluntarily endure discomfort. Canine procedures in particular often require forceful or sedated intervention, and may still leave the patient in discomfort.

### SUMMARY

An oxygen catheter for veterinary patients includes an integrated catheter, cannula or vessel having integrated lumens for both oxygen and anesthetic such as lidocaine. The oxygen lumen extends into the trachea of the veterinary patient, often a canine, for respiratory aid. The anesthetic lumen is integrated into the same catheter as the oxygen, and terminates prior to the oxygen lumen for administering an anesthetic such as lidocaine around the soft palate to ease discomfort of the inserted catheter. The integration of the respiratory and anesthetic vessels allows a lidocaine drip to accompany the catheterized oxygen supply to medicate the tracheal, pharynx and soft palate regions from the inserted catheter along the path of the oxygen lumen to ease patient discomfort and anxiety for effective respiratory treatment while avoiding more invasive and expensive measures.

Configurations herein are based, in part, on the observation that veterinary practitioners have a need for supplemental respiratory assistance for effective treatment of patients. Unfortunately, conventional approaches to veterinary medicine suffer from the shortcoming that bioethics and resource allocations differ for veterinary patients. Patient comfort and cost may be subject to different standards than human treatment. Accordingly, configurations herein substantially overcome the cost and comfort impediments of conventional veterinary care by providing an integrated respiratory catheter that provides both an oxygen lumen and anesthetic lumen that provide respiratory support while easing discomfort with a low cost device.

A full treatment package based on configurations herein includes a harness with interconnecting straps for engaging a canine head, and a plurality of integrated tubular vessels conjoined in a parallel arrangement or bundle and attached to a strap on the harness. Each tubular vessel of the plurality of tubular vessels has a distal end for patient insertion and a proximate end for receiving a fluidic source (oxygen or lidocaine). An attachment wing attaches to the tubular bundle downstream of the proximate end just prior to patient insertion, generally adjacent a nostril in a canine application. An inverted bend in the integrated catheter facilitates insertion and placement into a canine nasal passage. Once inserted, a plurality of fenestrations at the distal end of each of the plurality of vessels provide for fluid delivery into the canine nasal passage, while a respective fluidic engagement port at the proximate end of each of the tubular vessels receives the oxygen and lidocaine for administration through the vessels.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a perspective view of the integrated respiratory catheter (catheter) and a transition to an articulated shape for patient administration;

FIGS. 2A-2D show various segments of the catheter of FIG. 1;

FIGS. 3A and 3D show a cross section of the catheter and integrated lumens;

FIG. 4 shows engagement of the catheter with external oxygen and medication sources using vessel connectors;

FIGS. 5A and 5B show views of the catheter following administration to a patent; and

FIGS. 6A-6B shows fenestration patterns in the oxygen administration segment.

### DETAILED DESCRIPTION

The description below presents an example configuration for an oxygen nasal catheter or cannula designed for canine patients that includes an additional chamber for the infusion of a local anesthetic (such as lidocaine) into the nasal cavity, a harness and suture wing to secure the cannula to the patient's face, and an integrated catheter connection adapter.

In the configurations discussed below, the catheter is defined by an integrated assembly of vessels appearing as a length of tubing or similar cannulated structure, meaning an elongated, flexible vessel for transporting oxygen or anesthesia. Each vessel in the integrated catheter has a lumen for fluid passage, generally closed except for a distal end where fenestrations or apertures provide delivery of the respective fluid along the segment defined by the fenestrations. Additional structural features and refinements are discussed below.

Veterinary medicine differs from human healthcare because a different regime of bioethics is recognized. Veterinary cost models do not exhibit the same standard of care and are more swayed by such cost factors. In conventional approaches, oxygen supplementation is primarily limited to those veterinary hospitals that can provide 24/7 care, usually in an intensive care unit (ICU). The current gold standard of oxygen supplementation is a climate-controlled enclosure that provides higher ambient oxygen levels—simply known as an oxygen cage. The primary benefit of the oxygen cage is the ability to deliver oxygen by non-invasive means. Unfortunately, oxygen cages are expensive and quite large. As a result, they are only found in large specialty and referral veterinary hospitals.

A conventional method of nasal oxygen delivery (for those patients that do not have access to an oxygen cage or simply cannot fit into one) is via a rubber catheter inserted into one or both nasal cavities (to the depth of the nasopharynx or trachea) and sutured to the patient's face, often on or near the sensitive nasal tissue, often imposing discomfort. The rubber catheter is then connected to oxygen tubing by a catheter adapter or arbitrary vessel connection.

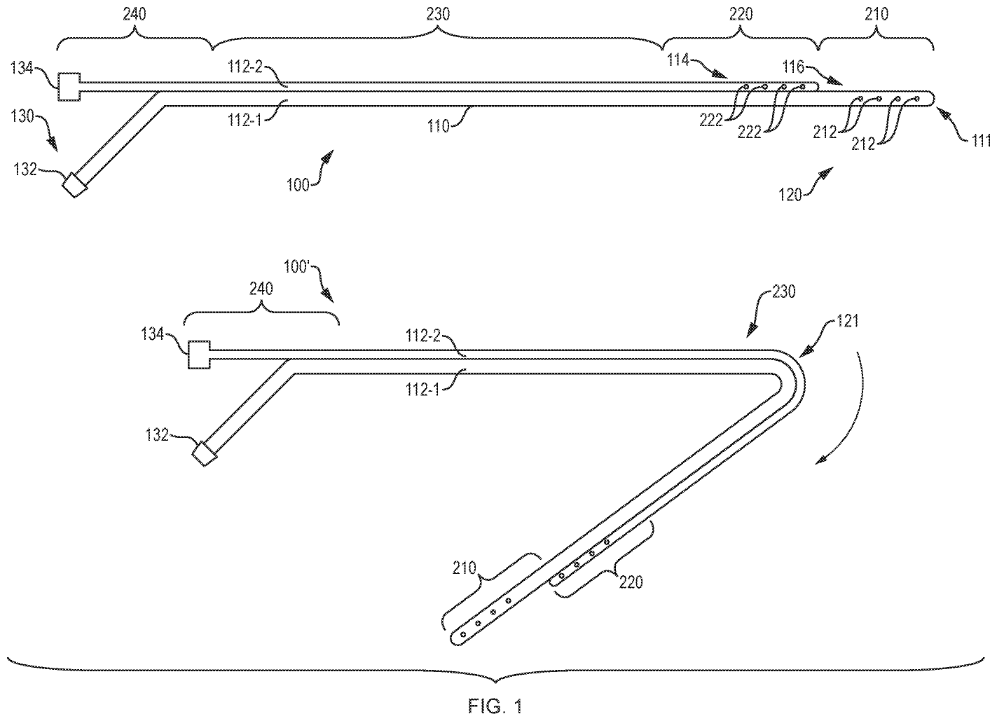

FIG. 1 shows a perspective view of the integrated respiratory catheter and a transition to an articulated shape for patient administration. Referring to FIG. 1, the disclosed veterinary respiration device 100 includes a respiratory catheter 110 having a plurality of integrated lumens 112-1 . . . 112-2 (112 generally) adapted for nasal insertion at a distal end 120. The integrated lumens 112 include an oxygen lumen 112-1 adapted for transport of a gaseous substance, typically oxygen or an oxygen mixture, and an anesthetic lumen 112-2 adapted for transport of a liquid anesthetic. For patient delivery, the anesthetic lumen 112-2 has an anesthetic passage 114 at the distal end 120, and the oxygen lumen 112-1 has an oxygen passage 116 more distal than the anesthetic passage. The oxygen passage 116 is generally at or adjacent a most distal point of the integrated catheter 110, and the anesthetic passages terminate prior, about 5-20 cm back from a terminus 111 at the distal end, to administer medication (anesthetic) to the sensitive tissue along which the catheter 110 extends.

The catheter 110′ has an inversion 121 or articulation for directing the integrated respiratory catheter 110 for nasal insertion. Inversion may occur either before or after patient nasal insertion, and occurs at a point for a proper depth of the distal end. The inversion should occur around the point of insertion so that the articulation and a small run of the catheter are outside the nostril, as the remaining portion towards the proximate end is adjacent to the jawline and skull of the canine patient. Variations on the degree and location of the articulation may occur based on an intended insertion depth based on patient anatomy.

At a proximate end 130, an oxygen source connection 132 is adapted to receive a respiratory oxygen source, typically from a nylon or flexible tube. The oxygen source connection 132 is configured to receive any suitable low to moderate pressure connection of respiratory air. A barbed fitting may be employed for frictionally engaging the source tubing with sufficient resistance to withstand any backpressure from a modest flow, typically around 5-10 liters/minute.

An anesthetic connection 134 engages the smaller diameter anesthetic lumen 112-2, and may be a needleless connector such as a Luer fitting, often employed for low pressure or drip fluid delivery.

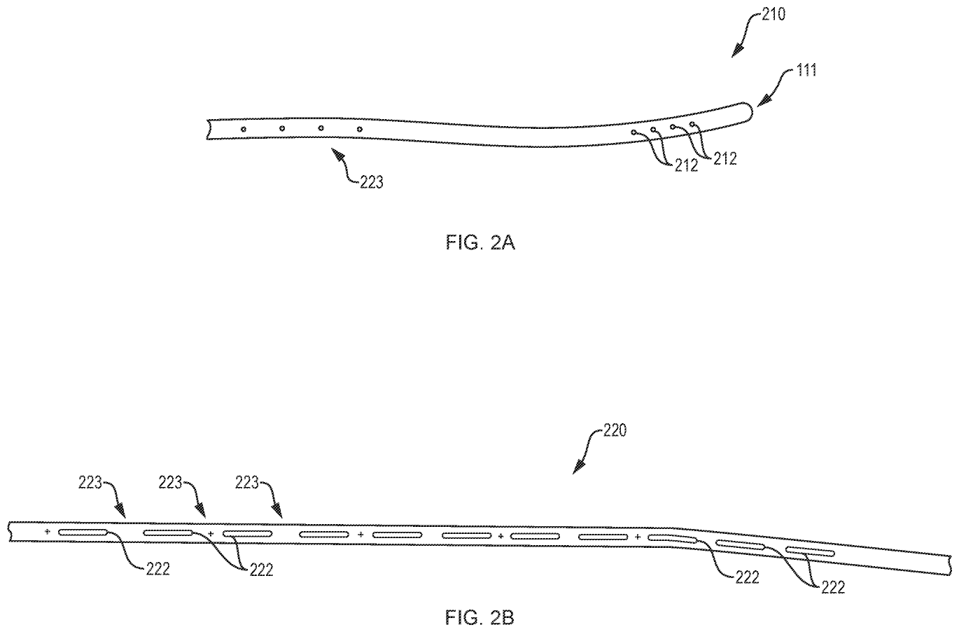

FIGS. 2A-2D show various segments of the catheter of FIG. 1. Referring to FIGS. 1 and 2A-2D, a gaseous (oxygen) administration segment 210 delivers the oxygen or other sourced respiration mixture from around the distal end 120 into the respiratory tract around the trachea and soft palate depending on the insertion depth, shown specifically in FIG. 2A. The gaseous administration segment 210 is at the end or terminus of the oxygen passage (lumen 112-1) and includes a sequence of fenestrations 212 at the distal end of the oxygen lumen. The sequence of fenestrations is generally one or more rows of holes or apertures with a collective area to pass sufficient oxygen to the patient. The tip 111 may be an opening with rounded edges to facilitate slidable insertion, or it may be a closed, semispherical structure which allows the fenestrations 212 to provide the O₂ flow. Also, the transported respiration mixture may include any suitable respiratory treatment, such as a gaseous anesthetic or vapor/humidification, as therapeutically called for.

An anesthetic administration segment 220 (FIG. 2B) is upstream from the gaseous administration segment 210, and includes fenestrations 222 from the anesthetic lumen 112-2 for administration of a liquid anesthesia to soothe and numb the sensitive tissue around the soft palate where the catheter 110 passes. The fenestrations 222 are adapted for passage of a viscous anesthetic, such as lidocaine, and have an elongated elliptical or oval shape. The fenestrations 222 are formed as a serial row at an outer circumferential wall, as determined by the integration and/or shared wall with the oxygen lumen 112-1. A typical medication would be a lidocaine drip, which flows slowly due to a viscous property, and the fenestrations 222 are positioned and adapted to deliver a slow, constant quantity to flow and coat the tissue adjacent to the catheter 110 to numb and soothe the sensitive tissue. The sequence of fenestrations 222 in the anesthetic lumen 112-1, precedes the sequence of fenestrations in the oxygen lumen 112-1, meaning the oxygen fenestrations 212 are closer to the distal end 120.

A length of the catheter 110 forms a transport segment 230 (FIG. 2D) for extending the lumens 112-1, 112-2 over the appropriate distance. A typical configuration covers a total length of about 40 cm, with the articulation 121 or “bend” around the midpoint to correspond to a near 180° turn into the nostril. A set of incremental markings 223 may be printed or rendered on the catheter to aid with insertion depth. It should be apparent that both lumens 112-1, 112-2 run parallel and conjoined along this segment. The anesthetic lumen 112-1 need extend only to the anesthetic administration segment 220.

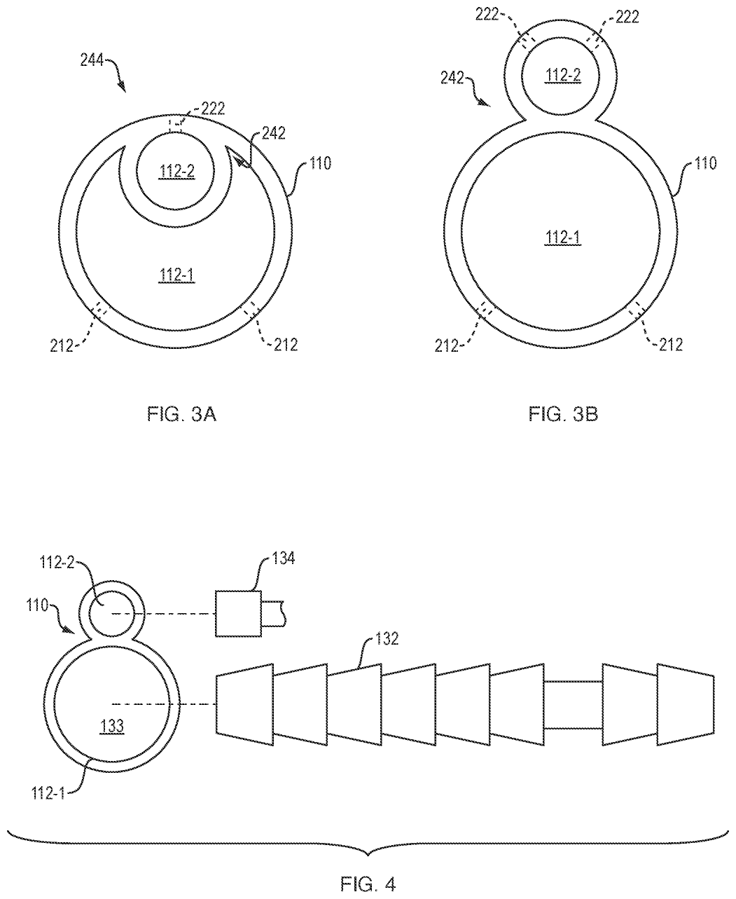

A supply segment 240, shown in FIG. 2C, includes ports, receptacles or engagement orifices for fluidic connection to sources for the oxygen and anesthetic. Here, the lumens 112 diverge into an oxygen source vessel 112′-1 and an anesthetic source vessel 112′-2. A respective port 132, 134 terminates the source vessels. Any detachable fluidic connection may be employed, discussed further in FIG. 4 below. Moderate pressure, such as a gravity or IV (intravenous) based flow, is amenable to the anesthetic supply, and a gaseous oxygen supply, a common utility in medical facilities, provides low pressure oxygen.

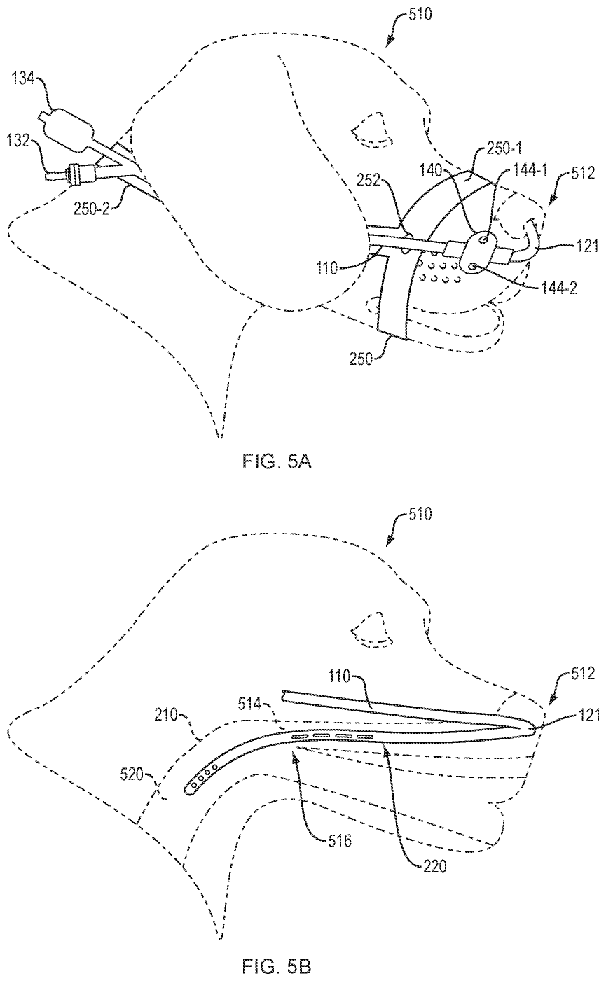

A multi-point attachment 140 has a slidable engagement 142 with the respiratory catheter 110, such that the slidable engagement is adapted for securement to a patient at a predetermined location on the respiratory catheter 110 based on a patient skeletal structure, typically the patient skull size and the location of a corresponding support strap or harness. The slidable engagement 142 may be a frictional, circumferential enclosure around the catheter 110, or may be a split, deformable pair of opposed flanking members biased around the circumference of the catheter 110. Slidable engagement allows the attachment 140 to be disposed along the catheter 110 to a suitable attachment position, discussed further below in FIGS. 5A and 5B. Two or more attachment apertures 144-1, 144-2 allow sutured or other fixation to the patient to be distributed, while the slidable engagement allows positioning away from sensitive nasal regions.

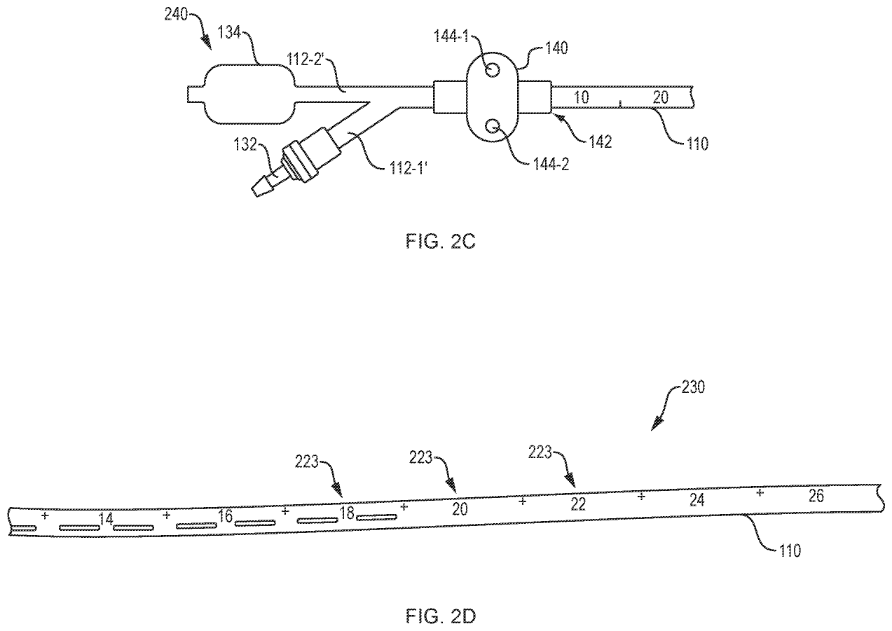

FIGS. 3A and 3B show a cross section of the catheter and integrated lumens. Referring to FIGS. 1-3B, the catheter 110 includes at least an oxygen lumen 112-1 for transport and administration of a gaseous medium, and an anesthetic lumen 112-2 for transport and administration of a fluid medium. Several configurations may be implemented, as long as each lumen has an outside portion in communication with the patient tissue/anatomy. Due to the respective gaseous and liquid mediums, the oxygen lumen 112-1 has a greater cross section area than the anesthetic lumen 112-2. In the configuration of FIG. 3A, the anesthetic lumen 112-2 occupies an interior portion of a circular cross section of the oxygen lumen 112-1, forming a “partial moon” or embedded circle shape. The fenestrations 222 in the anesthetic lumen 112-2 pass through the sidewall 244 common to both lumens 112. Oxygen fenestrations 212 may be at any outer wall not obscured by the anesthetic lumen 112-2.

In the configuration of FIG. 3B, the anesthetic lumen 112-2 is engaged at a tangent 242 to a circular cross section of the oxygen lumen 212, in a “stacked” or adjacency arrangement. The overall outer contour has a figure-8 form, however the fenestrations 222 may occupy a larger radii of the lumen 112-2 outer wall and may form multiple rows. The FIG. 3A configuration also engages at a common tangent 242 to the circular form of both lumens 112, however the anesthetic lumen extends inward from the tangent rather than outward.

In either construction, the respiratory catheter 110 is formed from a flexible outer wall, such that the flexible outer wall permits articulation to form the inversion 121 while avoiding an interrupting crease that impedes fluid flow. A nylon tubing structure may be employed for having a suitable rigidity and deformity.

FIG. 4 shows engagement of the catheter with external oxygen and medication sources using vessel connectors. In an example configuration, the oxygen lumen 112-1 may define a receptacle 133 adapted for receiving a barbed fitting 132 at the proximate end 130 of the oxygen lumen. A dual barb fitting 132 is shown; other suitable connectors or ports may be molded or attached for fluidic connection. Similarly, a needleless adapter 134 such as a Luer fitting may be included for receiving an anesthetic fluid at the proximate end 130 of the anesthetic lumen 112-2. Luer fittings are popular for delivery of liquid medications, however other suitable repositories may be engaged for an anesthesia supply.

FIGS. 5A and 5B show views of the catheter following administration to a patent. Referring to FIGS. 1-5B, a canine patient 510 exhibits an elongated nasal/jaw structure suitable for demonstrating advantages of the disclosed approach. A distance from the nostril to the trachea is large relative to the overall skull. A harness 250 includes a snout strap 250-1 extending around the snout around the rearward jaw just short of the eyes, to avoid interfering with modest jaw opening. A cranial strap 250-2 extends from the snout strap 250-1 to loop behind the skull and retain the harness 250 from falling off the tapered snout. One or more retentions 252 secure the catheter 110 to the harness 250.

In operation, the harness 250 supports the catheter 110 in a suitable position along the patient skull. The harness 250 provides a tethered engagement to the respiratory catheter 110 using a circumferential strap for patient cranial placement. Typically, the catheter is attached adjacent to the nostril entry point. Often this is a sutured connection, and conventional approaches employ a braid, trap or other tethered connection to a single suture point on the sensitive nasal exterior. This has the effect of focusing all the attachment force on one location, which can increase discomfort. In the claimed configuration, the respiratory catheter 110 has a multi point attachment at an intermediate portion of the respiratory catheter, on the proximate side of the articulation 121. The multi point attachment includes a plurality of binding locations defined by attachment apertures 144 of the attachment 140 for tethered securement of the integrated respiratory catheter 110 to the patient 510. The apertures 144 may engage sutures attached rearward of the nasal epidermal tissue 512, on a fur region. The use of multiple suture locations aids in dispersing any tension in the sutures, easing discomfort. The inserted catheter 110 passes from the nostril through the pharynx 514, past the soft palate region 516 for communication with the trachea 520.

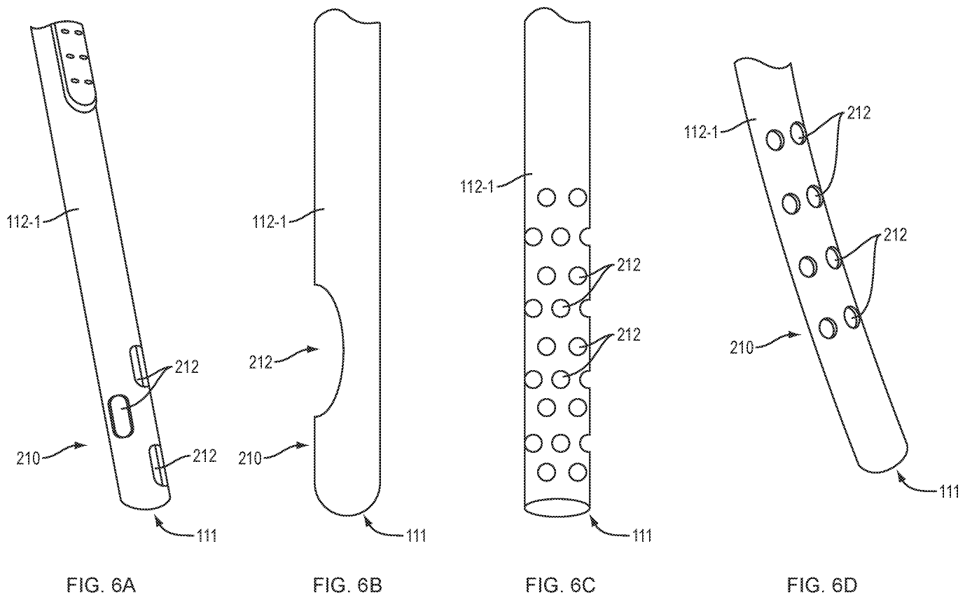

FIGS. 6A-6B shows fenestration patterns in the oxygen administration segment. Any suitable vessel and fenestration arrangement may be employed for the gaseous administration segment 210 and the anesthetic administration segment 220. Referring to FIGS. 6A-6B, varied arrangements of fenestrations 212 from the oxygen lumen 112-1 are illustrated. FIG. 6A shows curved rectangular fenestrations near a closed terminus 111. FIG. 6B shows a single large fenestration adjacent a closed terminus 111. FIG. 6C shows staggered rows of fenestrations 212 approaching an open vessel at the terminus 111. FIG. 6D shows rows of fenestrations 212 aligned axially and approaching an open terminus 111.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A veterinary respiration device, comprising:
a respiratory catheter having a plurality of integrated lumens in the respiratory catheter and adapted for nasal insertion at a distal end, the integrated lumens including:
an anesthetic lumen adapted for transport of a fluid anesthetic;
an oxygen lumen adapted for transport of a gaseous substance;
the anesthetic lumen having an anesthetic passage at the distal end; and
the oxygen lumen having an oxygen passage more distal than the anesthetic passage.

2. The device of claim 1 wherein the respiratory catheter has a multi point attachment at an intermediate portion of the respiratory catheter, the multi point attachment having a plurality of binding locations for tethered securement of the integrated respiratory catheter to a patient.

3. The device of claim 2 wherein the multi-point attachment has a slidable engagement with the respiratory catheter, the slidable engagement adapted for securement to a patient at a predetermined location on the respiratory catheter based on a patient skeletal structure.

4. The device of claim 1 wherein the catheter has an inversion for directing the integrated respiratory catheter for nasal insertion.

5. The device of claim 1 wherein the oxygen lumen has a greater cross section area than the anesthetic lumen, the anesthetic lumen occupying an interior portion of a circular cross section of the oxygen lumen.

6. The device of claim 1 wherein the oxygen lumen has a greater cross section area than the anesthetic lumen, the anesthetic lumen engaged at a tangent to a circular cross section of the oxygen lumen.

7. The device of claim 1 wherein the anesthetic passage includes a sequence of fenestrations in the anesthetic lumen, and the oxygen passage includes a sequence of fenestrations at a distal end of the oxygen lumen.

8. The device of claim 7 wherein the sequence of fenestrations in the anesthetic lumen precedes the sequence of fenestrations in the oxygen lumen.

9. The device of claim 7 wherein the anesthetic lumen terminates prior to the sequence of fenestrations in the oxygen passage.

10. The device of claim 1 further comprising:
a receptacle adapted for receiving a barbed fitting at a proximate end of the oxygen lumen; and
a needleless adapter for receiving an anesthetic fluid at a proximate end of the anesthetic lumen.

11. The device of claim 1 further comprising a tethered engagement to the respiratory catheter, the tethered engagement secured to a circumferential strap for patient cranial placement.

12. The device of claim 4 wherein the respiratory catheter is formed from a flexible outer wall, the flexible outer wall permitting articulation to form the inversion while avoiding an interrupting crease that impeded fluid flow.

13. A method for providing veterinary respiration, comprising:
disposing a respiratory catheter having a plurality of integrated lumens and adapted for nasal insertion at a distal end, the integrated lumens including:
an anesthetic lumen adapted for transport of a liquid anesthetic;
an oxygen lumen adapted for transport of a gaseous substance;
the anesthetic lumen having an anesthetic passage at the distal end, and
the oxygen lumen having an oxygen passage more distal than the anesthetic passage;
supplying an oxygenated respiration source via the oxygen lumen; and
supplying an anesthetic fluid via the anesthetic lumen.

14. The method of claim 13 further comprising securing the respiratory catheter via a multi point attachment at an intermediate portion of the respiratory catheter, the multi point attachment having a plurality of binding locations for tethered securement of the integrated respiratory catheter to a patient.

15. The method of claim 13 wherein the oxygen lumen has a greater cross section area than the anesthetic lumen, the anesthetic lumen engaged at a tangent to a circular cross section of the oxygen lumen.

16. A respiration device, comprising:
a harness including interconnecting straps for engaging a canine head;
a plurality of tubular vessels, the tubular vessels conjoined in a parallel bundle and attached to a strap on the harness, each tubular vessel of the plurality of tubular vessels having a distal end and a proximate end;
an attachment wing attached to the tubular bundle adjacent the proximate end;
the proximate end having an inverted bend for insertion into a canine nasal passage;
a plurality of fenestrations at the proximate end of each of the plurality of vessels for fluid delivery into the canine nasal passage; and
a fluidic engagement port at the distal end of each of the tubular vessels for receiving the fluid.

17. The device of claim 16 wherein the fenestrations are based on a type of fluid delivered via the tubular vessels.

18. The device of claim 16 wherein the attachment wing has a planar surface adapted for securement to a epidermal surface of the patient.

19. The device of claim 16 wherein the tubular vessels in include an oxygen vessel having a plurality of fenestrations for oxygen delivery.

20. The device of claim 19 wherein the tubular vessel include an anesthetic vessel having at least one fenestration adapted for delivery of a viscous anesthetic.
